(19) United States
(12) Patent Application Publication
(10) Pub. No.: US 2009/0325909 A1
(43) Pub. Date: Dec. 31, 2009

(54) Suppression of HIV replication and prevention and treatment of HIV

(75) Inventors: Mary C. Zink, Ellicott City, MD (US); Sheila A. Barber, Walkersville, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 10/569,887

(22) PCT Filed: Sep. 27, 2004
(86) PCT No.: PCT/US04/31747

§ 371 Date: Sep. 18, 2007

Related U.S. Application Data
(60) Provisional application No. 60/506,241, filed on Sep. 26, 2003.
(60) Provisional application No. 60/605,259, filed on Aug. 27, 2004.

(51) Int. Cl. A61K 31/65

(52) U.S. Cl. 514/154; 514/152

(57) ABSTRACT

The invention provides a new and effective treatment for human immunodeficiency diseases, particularly for HIV-infected individuals. The treatment utilizes tetracycline analogs, particularly minocycline, in amounts that are effective to prevent HIV replication both the central nervous system and in peripheral blood.

### RELATED APPLICATIONS

This application claims benefit of U.S. Provisional Patent Application No. 60/506,241 filed Sep. 26, 2003 and U.S. Provisional Patent Application No. 60/605,259 filed Aug. 27, 2004, the entire contents of which are incorporated herein by reference.

This invention was supported in part by grants MS-36911 and MH-069116 from the National Institutes of Health. The United States Government has certain rights in the invention.

### BACKGROUND OF THE INVENTION

1. Field of the Invention

The present invention relates to the field of medical arts; more particularly to the use of minocycline and related tetracycline compounds for treatment of HIV infections.

2. Background

Human Immunodeficiency Virus (HIV)

Human immunodeficiency-virus (HIV) central nervous system (CNS) disease is characterized by infiltration and activation of macrophages and microglia, production of proinflammatory cytokines, expression of proapoptotic and neurotoxic mediators and neuronal loss. HIV infection of the CNS often results in the development of cognitive, motor, and behavioral dysfunction known as HIV-associated dementia (Wilkie, et al., Aids 6:977-981 (1992). The classical neuropathological finding in HIV-1 infection of the brain is the presence of productively infected macrophages in perivascular cuffs and in microglial nodules. There is also widespread activation of microglia, reactive astrocytosis and evidence of neuronal loss and damage to the dendritic arbor (Everall, et al., J. Neuropathol. Exp. Neurol. 52:561-566 (1993) Although CNS inflammatory responses may be beneficial in the short term by containing virus replication, chronic CNS inflammation may cause damage through the generation of neurotoxic products. There is a close correlation between microgliaI and macrophage activation and the severity of HIV-induced neurological disease. (Glass, et al., Ann. Neurol., 38:755-762 (1995).

While many antiretroviral drugs suppress HIV replication in peripheral blood, few drugs achieve effective levels in the brain. In addition, many of the reverse transcriptase and protease inhibitors on the market are expensive, require complex dosing regimens and have significant side effects, including neurotoxicity. A number of neuroprotective agents for HIV-infected individuals are being examined in clinical trials. Most of these therapies are designed to inhibit the downstream effects of proinflammatory mediators or to augment neuronal function, but no single agent has emerged as a solution to both the inflammatory and neurodegenerative effects of HIV in the CNS.

Anti-HIV Agents

Several drugs have been used to treat HIV, including antiretroviral and protease agents, for example, AZT, ddC, and ddI antivirals. These agents, when used alone, frequently have limited success in AIDS patients with advanced disease where the immune system is severely compromised. While these drugs may promote some improvement on a temporary basis, it appears that AZT and similar anti-retroviral drugs may only reduce HIV replication in vivo to a limited extent. In fact these nucleoside analogs do not drastically reduce HIV replication in chronically infected cells, as shown in laboratory experiments (Pausa, C. D., et al., J. Exp. Med. 172:10351042 (1990).

HIV infection of the CNS often results in the development of cognitive, motor, and behavioral dysfunction known as HIV-associated dementia (Wilkie, et al., Aids 6:997-981 (1992)). The classical neuropathological finding in HIV-1 infection of the brain is the presence of productively infected macrophages in perivascular cuffs and in microglial nodules. There is also widespread activation of microglia, reactive astrocytosis and evidence of neuronal loss and damage to the dendritic arbor. Although CNS inflammatory responses may be beneficial in short term by containing virus replication, chronic CNS inflammation may cause damage through the generation of neurotoxic products.

HIV-associated dementia continues to be a major clinical problem despite the a availability of highly active anti-retroviral agents, since many of these antiretroviral agents do not cross the blood-brain barrier and therefore are limited to activity in the peripheral blood system. There is a growing body of evidence implicating host inflammatory processes as playing a role in the neuronal damage of HIV-associated dementia; however, the detailed interactions and relationship to CNS HIV infection remains to be elucidated.

Primates have provided the only model to date accepted as applicable to HIV human infections because of the similarity of SIV to HIV. The SIV/macaque model has provided important information on the pathogenesis of HIV-induced CNS disease because it recapitulates key features of HIV CNS infection, including the eventual development of encephalitis with active virus replication in the CNS, characteristic histopathological changes, psychomotor impairment and neurodegeneration. (Weed, et al., J. Neurovirol., (2002)) Unfortunately, the prolonged course of infection (years) and the relatively low incidence of SIV CNS disease in the classical SIV/macaque model-limits its usefulness to elucidate pathogenic mechanisms of HIV-associated dementia (HAD) that may be vulnerable to therapeutic intervention.

Tetracyclines

Several closely related tetracyclines have bacteriostatic antibiotic activity and are active against a wide range of bacteria. The best-known members of this family are oxytetracycline, tetracycline, demeclocycline, methacycline, doxycycline and minocycline. Recently, “chemically modified tetracycline” (CMT) compounds have been prepared and identified as having activity against cancer, arthritis, and osteoporosis (Golub, et al., Adv. Dent. Res., 12:12-26 (1998). The CMT analogs are designed to eliminate antibacterial activity by removal of a dimethylamino group from the A ring. Despite lacking antibacterial activity, the CMT compounds exhibit a variety of activities such as anti-inflammatory activity and ability to inhibit mammal-derived matrix metalloproteinases (MMPs). Antifungal activity (Liu, et al., Antimicrobial Agents and Chemotherapy, v. 46; 1447-1454 (2002)) has been reported.

Minocycline is a semisynthetic second generation tetracycline that readily crosses the blood-brain barrier. It is a safe and effective antibiotic that has been prescribed for years and is available in generic form. During the early 1990s, minocycline was shown in clinical studies to have anti-inflammatory properties and to be useful in the treatment of rheumatoid arthritis and osteoarthritis (Ryan, et al., Curr. Opin. Rheumatol., 8:238-247 (1996)). More recently minocycline has been demonstrated to play neuroprotective roles in animal models of Huntington's disease, Parkinson's disease, amyotrophic lateral sclerosis, multiple sclerosis, cerebral ischemia, and traumatic brain disease.

For many years, the search for effective anti-HIV drugs was constrained by having to evaluate anti-HIV activity in vitro. A study by Lemaitre, et al. (1991) found that tetracycline analogs protected CEM cells from HIV-induced cytopathogenicity. Unfortunately, despite the cytoprotective activity, the authors found that the compounds failed to prevent HIV replication. Apparently, an earlier study showed that doxycycline and chlortetracycline were active at high concentrations, but had the undesirable effect of inhibiting DNA polymerase β (Wondrak, et al, 1988). This was thought to explain why nontoxic (low) doses of minocycline would not be suitable for treating and preventing HIV replication, despite showing some in vitro cytoprotective activity (Lemaitre, et al., 1991), due to unacceptable toxicity to healthy cells in vivo.

In vitro studies conducted in 2002 reported inhibition of HIV replication macrophages, microglial cells and astrocytes by minocycline. Infection of cell cultures with R5 and X4R5-HIV, HIV-G-pseudotyped and VSV-G-reporter viruses inhibited HIV-1 replication at IC₅₀ of ˜20 μg/ml. Chronically infected infected U1 cells and LTR promoter activity in U38 cells were also inhibited (Si, et al., Program No. 395.9, 2002 Abstract Viewer/Iternary Planner, Washington, D.C., Society for Neuroscience, 2002). There was no evidence that the same effects could be obtained in vivo, or that HIV CNS could be successfully treated.

More recently, Berman (U.S. Pat. No. RE38,386E) has presented an extensive list of chemically modified tetracyclines (CMT) claimed to heal HIV viral infections. The extensive list included minocycline CMT; however, none of the compounds was tested either in vivo or in vitro, nor was it suggested that any of the compounds would actually prevent HIV replication or that any of the unmodified tetracyclines would show activity in healing HIV infections. Moreover, the CMT minocycline in the extensive list of proposed CMTs would, under the accepted definition of CMT, lack the 4-position dimethylamino, group, making it lose antibacterial activity, in contrast with minocycline, which has antibacterial activity.

Deficiencies in the Art

Although highly active anti-retroviral therapy (HAART) has reduced the incidence of clinical signs of neurological disease in HIV-infected individuals, autopsy studies suggest that there has been no corresponding decline in the incidence of inflammatory lesions in the CNS of HIV-infected humans. In addition, there is evidence that HAART has been less effective in lowering virus replication in the CNS than in the blood and HAART resistant viruses have been identified. Anti-HIV drugs have widely differing abilities to cross the blood-brain barrier and have different bio-availabilities in the brain; in fact most do not effectively cross the blood-brain barrier and therefore would not be expected to slow the progress of HIV-associated dementia.

Because several anti-retroviral drugs will suppress HIV replication in peripheral blood, there is some interest in modifying these drugs, or finding new drugs, that will achieve effective levels in the brain. It is believed that the brain is a significant reservoir for the virus such that HIV replication can lead to microglial activation, macrophage infiltration, production of pro-inflammatory cytokines, enhanced production of neurotoxic mediators and neuronal loss. No single agent is known to be effective in treating the inflammatory and neurodegenerative effects of HIV in the central nervous system and demonstrating an inhibition of HIV-replication in the brain. Accordingly, there is a need for therapeutic agents that are capable of crossing the blood-brain barrier and have viral suppressive and neuroprotective properties.

There is substantial evidence that the CNS constitutes a significant reservoir for virus in HIV-individuals despite anti-retroviral therapy. These observations emphasize the importance in developing novel therapeutic strategies to suppress HIV replication in the CNS and prevent the development of neuropathological changes.

### SUMMARY OF THE INVENTION

The invention is the discovery that minocycline, a semisynthetic analog of tetracycline, has significant activity in reducing SIV replication in the brain and directly suppressing neurotoxic effects in vivo. In particular, it has been found that, in contrast to previous studies, minocycline formulations are highly effective suppressants of SIV replication in the central nervous system and in the plasma of a significant portion of animals. The results in primate models are a strong indication that the disclosed compositions represent a major step forward in the arsenal of treatments for HIV-infected individuals.

The discovery that minocycline inhibits HIV replication in vivo is remarkable because the drug has historically been used to treat microbial infections and more recently been found useful in treating certain inflammatory diseases. The in vivo tests of minocycline are significant because the compound clearly inhibits replication of a highly aggressive form of HIV in both blood and the CNS in a primate animal model. Implications for human use are important because most other drugs for HIV have many disadvantages, including toxicity and the development of resistance to the therapy.

An important aspect of the invention therefore includes methods of treating or preventing HIV and related immunodeficiency virus infections in primates, particularly humans. The methods provide for administration of effective amounts of a tetracycline related compound, particularly minocycline, such that HIV replication is entirely or substantially inhibited. It is contemplated that minocycline will also be useful in treating other retroviral diseases, in addition to HIV, and may also prove effective against evolving strains of HIV resistant to current treatments.

A preferred embodiment of the invention is a method of suppressing HIV or a related HIV replication in a mammal. An amount of minocycline or a derivative thereof effective to suppress HIV replication in the mammal is administered. The mammal may be a primate such as a simian, but may also be species such as cows or cats. The HIV that infects primates is designated as SIV for simian immunodeficiency virus. It is analogous to the HIV infecting humans and has similar effects. Preferably the mammal to be treated is a human and treatments are directed to human subjects.

Treatments for HIV have historically lost effectiveness after use, sometimes as short as six months. Minocycline and other semi-synthetic tetracyclines may prove effective in “cocktails” or in serial administration against evolving a V strains. Examples of HIV strains include HIV-2ST, HIV-2ROD and may in some cases be less virulent than HIV-1; however, prevention of retroviral replication would be beneficial, particularly if some of the strains survived in the CNS and contributed to low-grade inflammation.

Human retroviruses include not only HIV-1 but also HIV-2, HTLV2 and HTLV1. HTLV1 causes T-cell leukemias and lymphomas, and while HTLV2 has no known human pathology, it is known to infect CD4 cells, as does HIV-1. In view of the several identified actions of minocycline, there is perhaps a benefit in considering early intervention in human retroviral infection, in order to prevent replication in the CNS where long-term damage may occur.

Other embodiments of the invention include pharmaceutical compositions comprising minocycline or a tetracycline analog or physiologically acceptable salts thereof in an amount that is effective to suppress retroviral replication in brain and peripheral locations when administered to HIV/SIV-infected mammals. Preferably the mammals are HIV-infected humans.

It is also contemplated that any of a number of therapeutic agents may be administered with minocycline, depending on the state of health of the patient. Additional agents include, but would not be limited to, additional antibiotics, anti-inflammatory agents, anti-HIV drugs or antifungal compounds and combinations thereof.

When employed as a single agent, the amount of minocycline delivered is preferably on the order of 0.5-25 mg/kg per day, more preferably in the range of 1-10 mg/kg/day and most preferably about 4 mg/kg/day. These amounts will of course be adjusted depending on the age, race, sex and health of the individual treated, in addition to any other drugs that may be co- or subsequently administered.

The methods of the invention are directed to administering to a subject in need thereof, particularly a human subject, an effective amount of minocycline or other tetracycline analog, optionally in combination with other therapeutic agents. Such methods involve a step of identifying that the subject is in need of such treatment; that is, has AIDS or is HIV-positive, although it is possible that a qualified professional may identify a subject who has been clearly exposed to HIV for prophylactic treatment. An example may be a rape victim or members of populations where AIDS is rampant. Identification of a subject in need thereof can be in the judgment of the subject to some extent, but in general should be made by a health professional as measured by standard tests for HIV. Such tests are available commercially and can be performed in various health facilities or in the field. Identification normally includes taking a sample from the candidate subject and analyzing the sample, which step then assists the professional in determining whether or not treatment is to be initiated.

Treatments will be indicated for virtually any subject who is HIV-positive; however, it will be appreciated that the methods should also be applied to those who have fall-blown indications of AIDS because of the disclosed effect of the minocycline formulations in the inhibition of HIV replication in both plasma and brain. In rare instances, for example in HIV-negative subjects, the inventive treatment may be initiated. Such cases may include those individuals known to have been exposed to HIV in populations where HIV is endemic, and those who present with encephalitis suspected to have a viral etiology. It is believed that such prophylactic treatment is safe because minocycline and other tetracycline analogs are safe, effective, and are increasingly recognized as effective in treating and possibly preventing a wide variety of seemingly unrelated diseases such as inflammatory conditions.

The invention particularly includes pharmaceutical preparations of minocycline, which are well-known and can be prepared for oral, intraveneous, interperitoneal or topical administration. Ono, et al. (U.S. Pat. No. 6,566,350, May 20, 2003) for example, has disclosed aminocycline composition for topical administration, which may be a preferred form for disabled patients or for use in rapidly treating highly HIV-infected sub-populations. Oral formulations are especially preferred because of convenience and, in the case of minocycline, have well-documented absorption and blood level profiles. Oral formulations may be liquid or solid; for example, in tablet form. Solid formulations are ideal for storage, shipping and easy distribution for example in third world countries with remote populations in need of HIV therapy.

Use of the invention is believed to be quite general and is expected to be beneficial not only to those individuals who are asymptomatic, but who are HIV-positive, but also to those with more progressed disease having symptoms associated with AIDS.

### BRIEF DESCRIPTION OF THE DRAWINGS

FIG. 1A. Numerous epitheloid macrophages and multinucleated giant cells in perivascular spaces are seen. Minocycline reduces the severity of SIV encephalitis. Representative microscopic findings in an SIV-infected, untreated macaque with severe encephalitis.

FIG. 1B shows that the changes seen in FIG. 1A are absent in brain tissue from a macaque treated with minocycline H & E×200

FIG. 2A. Minocycline suppresses expression of markers of MHC Class II, a marker of CNS inflammation.

FIG. 2B shows minocycline suppression of CD68, used to detect macrophages and microglia

FIG. 2C shows the effect of minocycline on TIA-1, used to identify cytotoxic lymphocytes

FIG. 2D shows the effect of minocycline on p-p38 to detect activation of p38. FIGS. 2A-2D indicate macaques with severe encephalitis □; moderate encephalitis O; mild encephalitis ▪ and no encephalitis .

FIG. 3A shows minocycline suppression of expression of MCP-1 in the CNS of untreated-macaques. MCP-1 protein expression in the CSF, depicted as the ratio of MCP-1 in the CSF relative to plasma measured by ELISA at each time point; severe encephalitis □; moderate encephalitis O; mild encephalitis ▪ and no encephalitis .

FIG. 3B shows minocycline suppression of expression of MCP-1 in the CNS of individual macaques; Not identified are points represented by untreated macaque CC33, BM03, BI55, BP41, BK09, BP33, and treated macaques 01P010, CT63, CT4A, CT1C, and CT1F. Arrows indicate when minocycline was initiated.

FIG. 3C shows the MCP-1 protein quantified in brain homogenates by ELISA where symbols represent macaques with severe encephalitis □; moderate encephalitis O; mild encephalitis ▪ and no encephalitis (indicating mean of all macaques in group).

FIG. 4A shows viral gene expression in the CNS of untreated macaques. SIV RNA was quantified by real-time RT-PCR from virus isolated from the CSF of SIV-infected untreated and minocycline-treated macaques. The different symbols represent CC33, BM03, and BI55.

FIG. 4B shows that minocycline suppresses viral gene expression in the CNS. SIV RNA was quantified by real-time RT-PCR from virus isolated from the CSF of SIV-infected untreated and minocycline-treated macaques. The different symbols represent BP41, BK09, BP33, 01P010, CT63, CT4A, CT1C, and CT1F. Arrow indicates when minocycline was initiated.

FIG. 4C quantifies total RNA isolated from basal ganglia. Viral protein in the brain was quantified by digital, image analysis and brain sections from SIV-infected, untreated and minocycline-treated macaques immunohistochemically stained for viral antigen gp41. Symbols represent macaques with severe encephalitis □; moderate encephalitis O; mild encephalitis ▪ and no encephalitis .

FIG. 4D shows gp41 expression for macaques with severe encephalitis □; moderate encephalitis O; mild encephalitis ▪ and no encephalitis (mean for all macaques in group).

FIG. 5A shows minocycline suppression of SIV replication in primary PBL. Virus replication was quantified by p27 (SIV) EL1SA in supernatants collected from cultures of primary macaque PBL infected with SIV/DeltaB670.

FIG. 5B shows minocycline suppression of SIV replication in primary PBL. Virus replication was quantified by p27 (SIV) ELISA in supernatants collected from cultures of primary macaque PBL infected with SIV/17E-Fr.

FIG. 5C shows minocycline suppression of SIV replication in primary macrophages. Virus replication was quantified by p27 (SIV) ELISA in supernatants collected from cultures of primary macaque macrophages infected with SIV/DeltaB670.

FIG. 5D shows minocycline suppression of SIV replication in primary macrophages. Virus replication was quantified by p27 (SIV) ELISA in supernatants collected from cultures of primary macaque macrophages infected with SIV/17E-Fr.

FIG. 5E shows minocycline suppression of HIV replication in primary PBL. Virus replication was quantified by p27 (SIV) ELISA in supernatants collected from cultures of human PBL infected with IV-1 IIIB

FIG. 5F shows minocycline suppression of SIV replication in primary human macrophages. Virus replication was quantified by p27 (SIV) ELISA in supernatants collected from cultures of primary macrophages infected with HIV-Ba-L that had been pretreated for 24 hour with the indicated doses of minocycline and infected with the indicated viruses. Symbols represent the following concentrations of minocycline: (⋄ none, □ 10 μg/mL, Δ 30 μg/mL 40 μg/Ml).

FIG. 5G shows a Western blot analysis of total p38 and activated p38 (p38) in cultures of primary human PBL treated with different concentrations of minocycline.

FIG. 5H shows macrophages that had been pretreated for 24 hour with the indicated doses of minocycline (see FIG. 5F) demonstrating that treatment with minocycline suppresses activation of p38 in HIV-7 infected primary human PBL but not macrophages.

FIG. 6A is a pERK graphical expression in uninfected and SIV-infected macaques days post inoculation.

FIG. 6B shows astrocytes stained for pERK at 10 days.

FIG. 6C shows perivascular macrophages and multinucleated giant cells stained for pERK.

FIG. 6D is a pJNK graphical expression post inoculation as shown by immunostained brain sections from uninfected and SIV-infected macaques

FIG. 6E shows neuronal expression of pJNK in brain section sections from uninfected and SIV-infected macaques.

FIG. 6P shows staining of pJNK in macrophage cells at 84 days p.i.

FIG. 6G is a graphical expression of p38 post inoculation of macaques with SIV.

FIG. 6H shows staining of neurons at 84 days.

FIG. 6I shows staining of astrocytes at 84 days.

FIG. 7 Comparative expression of pERK (neuroprotective) vs. pJNK and p-p38(neurodegenerative) in brains of macaques during acute (10 days p.i.), asymptomatic (21 and 56 days p.i.) and terminal (84 days p.i.) infection. Bars represent the net effect of neuroprotective and neurodegenerative influences based on percent change in expression of these signaling molecules, assuming that pERK, pJNK and p-p38 have equal and independent influences. During acuteinfection, there was a net increase in expression of pERK, whereas during terminal infection, neurodegenerative pathways, particularly p-p38, predominated.

### DETAILED DESCRIPTION OF THE INVENTION

The demonstration that a well-tolerated, safe antibiotic agent can prevent HIV/SIV replication in the CNS and peripheral blood in vivo is a significant discovery. This fulfills a need for agents that have different and broader activities over currently used regimens for HIV and AIDS treatments. The invention is expected to provide a new therapy as a single component agent and in combination with other similar drugs or agents that can be used to concurrently treat conditions related to HIV or arising because of the insult to the immune system.

Minocycline [4S-(4a,4aa,5aa,12aa)-4,7-bis(dimethylamino)-1,4,4a,5,5a,6,11,12a-octahydro-3,10,12,12a-tetrahydroxy-1,11-dioxo-2-naphthacene carboxamide]monohydrochloride has the structural formula C₂₃H₂₇N₃O₇.HCl. It is a semi-synthetic second generation tetracycline that readily crosses the blood-brain barrier (Brogden, et al., Drugs, 9:251-291 (1975)) and is a safe and effective antibiotic that has been prescribed for years and is available in generic form.

During the early 1990's, minocycline was shown in clinical studies to have anti-inflammatory properties and to be useful in the treatment of rheumatoid arthritis and osteoarthritis. More recently, minocycline has been demonstrated to play neuroprotective roles in animal models of Huntington's disease, Parkinson's disease, amylotrophic lateral sclerosis, multiple sclerosis, cerebral ischemia, and traumatic brain disease. It has also been evaluated in human trials for Huntington's disease (Bonner, et al., Science, 278:1481, 1483 (1997).

Investigations into the mechanism(s) by which minocycline protects neurons from cell death have suggested that the drug alters a variety of pathways in microglial cells. Of particular interest is the pathogenesis of HIV CNS disease as described in studies demonstrating the ability of minocycline to inhibit microglial cell-mediated excitotoxic damage to neurons (Battanai, Pharmacol. Res., 44:353-361 (2002)). This differentiates minocycline from other neuroprotective agents such as CPI-1189, which exerts its effects on cytokine-induced alterations in the brain.

Glutamate is an excitotoxin when present in excess and the glutamate receptor agonist kainite, when added to mixed cultures of neurons and microglial cells induce microglial proliferation and increase the release of NO and IL-1β. These responses are inhibited by minocycline. The results suggest that minocycline acts by inhibiting both proliferation and activation of microglia. Additionally, these and other studies demonstrated that minocycline also inhibited NO-induced activation of p38 in microglial cells. Levels of iNOS, IL-1 and/or activated p38 MAPK are also significantly decreased in minocycline-treated animals with ischemic brain damage, in minocycline-treated mice injected with MPTP (a model of Parkinson's disease) and in a transgenic mouse model of Huntington's disease. Decreased numbers of activated microglial cells in the brains of 6-hydroxydopamine injected mice (model of Parkinson's disease) treated with minocycline as compared with injected untreated mice have also been reported.

Minocycline has been reported to reduce the levels of activated caspase-1 and caspase-3 in the brains of mice subjected to traumatic brain-injury, in a rat model of hypoxemia-ischemia and in a transgenic mouse model of Huntington's disease (Acarin, et al., J. Neurosci. Res., 68:745-754 (2002). It was also found that minocycline inhibits expression of matrix metaloproteinases 2 and 9 and delays or abrogates the development of neurological signs in EAE, a mouse/rat model of multiple sclerosis (Adamson, et al., Science, 274:1917-1921 (1996)). This mechanism may be of particular relevance to the pathogenesis of HIV CNS disease, in consideration of data demonstrating an increase in MMP-9 in the brains of HIV-infected individuals with dementia and recent data demonstrating that S-nitrosylation of MMP-9, along with oxidative changes may play an important role in the induction of neuronal cell death (Arvin, et al., Ann. Neurol., 52:54-61 (2002).

The observation that minocycline inhibits neuronal death in such a wide variety of in vitro and in vivo models suggested that it might inhibit a critical step in the execution of a common death pathway. Recent studies had demonstrated that minocycline inhibits cytochrome c release from the mitochondria into the cytoplasm in response to excitotoxic (glutamate) stimuli. The release of cytochrome c across the outer mitochondrial membrane into the cytoplasm is a central step in apoptotic pathways. The neuroprotective effects of minocycline in a wide variety of neurodegenerative diseases, revealing its ability to suppress macrophage activation and proliferation, taken together suggested the importance of examining the potential role of minocycline in suppressing the inflammatory changes in HIV/SIV CNS disease.

CNS disease is a frequent complication of BV-1 infection. It was believed that identification of cellular mechanisms that control virus replication and that mediate development of HIV-associated neuropathology would provide novel strategies for therapeutic intervention. The milieu of the CNS during HIV infection is extraordinarily complex due to infiltration of inflammatory cells and production of chemokines, cytokines and neurotoxic moieties. Cells in the CNS must integrate signaling pathways activated simultaneously by products of virus replication and infiltrating immune cells. Activation of mitogen-activated protein kinases (MAPKs) in the CNS of SIV-infected macaques during acute, asymptomaric and terminal infection was investigated.

It was shown that significantly increased (p<0.02) activation of ERK MAPK, typically associated with anti-apoptotic and neuroprotective pathways, occurs predominantly in astrocytes and immediately precedes suppression of virus replication and macrophage activation that occur after acute infection. In contrast, significantly increased activation of pro-apoptotic, neurodegenerative MAPKs JNK (p=0.03; predominantly in macrophages/microglia), and p38 (p=0.03; predominantly in neurons and astrocytes) after acute infection correlates with subsequent resurgent virus replication and development of neurological lesions. This shift from classically neuroprotective to neurodegenerative MAPK pathways suggested that agents that inhibit activation of JNK/p38 would be protective against HIV-associated CNS disease.

Although highly active antiretroviral therapy (HAAPT) has reduced the incidence of clinical signs of neurological disease in HIV-infected individuals, autopsy studies suggest that there has been no corresponding decline in the incidence of inflammatory lesions in the CNS. In addition, there is evidence that HAART has been less effective in lowering virus replication in the CNS than in the blood and HAART resistant viruses have been identified. Anti-HIV drugs have widely differing abilities to cross the blood-brain barrier and have different bioavailabilities in the brain.

Primate Model for CNS infection. SIV infection of macaques provides an excellent model to investigate the cellular mechanisms that control acute virus replication in the CNS and lead to neurological disease in HIV-infected people. SIV-infected macaques develop AIDS and neuropathological changes similar to those of HIV-infected individuals, including motor and cognitive deficits as well as multifocal and perivascular aggregates of brain macrophages and multinucleated giant cells, which serve as the major hosts for productive replication of virus in the CNS (Weed, et al. J. Neurovirol, (2002).

The SIV macaques model is an ideal system in which to evaluate therapeutic compounds such as minocycline because it recapitulates key features of HIV CNS infection, including the development of encephalitis with active virus replication in the CNS, characteristic histopathological changes, psychomotor impairment and neurodegeneration (Zink, et al., J. Virol. 73:10408-10488 (1999). Macaques can be infected with well-characterized strains of virus, blood, CSF and tissue samples can be obtained at regular intervals throughout infections, macaques can be treated with therapeutic doses of drugs on a known timetable, and they can be euthanized at defined states of disease to examine virus-replication and host responses. In the accelerated, consistent SIV/macaque model of HIV CNS disease over 90% of infected animals develop encephalitis with neurodegeneration. This model recapitulates the acute, asymptomatic, and terminal stages of HIV infection in humans on a highly reproducible time schedule. Use of this model has demonstrated that the development of SIV encephalitis coincides with a signaling imbalance favoring activation of pro-apoptotic (JNK/p38) pathways over antiapoptotic (ERK) MAPK signaling pathways (Barber, et al., Am. J. Pathol., 2003).

Minocycline Analogs. While the invention has been demonstrated with minocycline, it is apparent that closely related compounds are expected to have similar. HIV-inhibiting activities as minocycline. Such compounds may include tetracycline derivatives and analogs as well as the so-called “CMT” tetracyclines prepared by chemically modifying tetracycline. Among the many such CMT compounds, of tetracycline are: 4-dedimethylaminotetracycline; 4-dedimethylamino-oxytetracycline; 4-dedimethylamino-7-chlortetracycline; 4-hydroxy-4-dedimethylaminotetracycline; 5a,6-anhydro-4-hydroxy-4-dedimethylaminotetracycline; 6α-deoxy-5-hydroxy-dedimethylaminotetracycline; 6-dimethyl-6-deoxy-4-dedimethylaminotetracycline; 4-dedimethylamino-11-hydroxy-12a-deoxytetracyclines; 12a-deoxy-4-deoxy-4-dedimethylaminotetracycline; 6-alpha-deoxy-5-hydroxy-4-dedimethylaminodoxycycline; 12a,4a-anhydro-4-dedimethylaminotetracycline; 7-dimethylamino-6-demethyl-6-deoxy-A dedimethylaminotetracycline; 6a-benzylthiomethylenetetracycline; tetracyclinonitrile; mono-N-alkylated tetracycline amide; 6-fluoro-6-demethyltetracycline; 11a-chlortetracycline; tetracycline pyrazole; 12a-deoxytetracycline; 4-dedimethylamino-5-oxytetracycline; 5a,6-anhydro-4-hydroxy-4-dedimethylminotetracycline; 12a,4a-anhydro-4-dedimethylaminotetracycline; tetracyclonitrile; 7-chloro-4-dedimethylaminotetracycline; 12a-deoxy-4-deoxy-4-dedimethylaminotetracycline; 4-dedimethylamino-7-chlorotetracycline; 4-dedimethylamino-7-dedimethylaminotetracycline; 2-nitrilo analogs of tetracycline; 4-dedimethylamino 12a-deoyotetracycline; tetracyclines altered at the 2-carbon position to produce a nitrile; 4-dedimethylamino-7-chlorotetracycline; 6alpha-deoxy-5hydroxy-4-dedimethylaminotetracycline; tetracyclonitrile; 6-alpha-benzylthiomethyltetracycline; 11-alpha-chlorotetracycline; 7-chlortetracycline; 5 hydroxytetracycline; 6-demethyl-7-chlortetracycline; 6-demethyl-6-deoxy-5-hydroxy-6-methylenetetracycline; 6-alpha-benzylthiomethylenetetracycline-mono-N-alkylated tetracycline amide; 2-acetyl-8-hydroxy-1-tetracycline; 6-demethyl-6-deoxytetracycline; 6-demethyl-6-deoxy-5-hydroxy-6-methylenetetracycline; 2-acetyl-8-hydroxyl-1-tetracycline; 4-hydroxy-4-dedimethylaminotetracycline; 5a,6-anhydro-4-hydroxy-4-dedimethylaminotetracycline; 6-demethyl-6-deoxy-4-dedimethylaminotetracycline; 6-deoxy-8-demethyl-4-dedimethylaminotetracycline; 6a-deoxy-5-hydroxy-4-dedimethylaminotetracycline; pyrazole derivative of tetracycline; 7-chloro-6-demethyl-4-dedimethylaminotetracycline; 11-alpha-chlortetracycline; 4-dedimethylamino-7-chlortetracycline; 4-de(dimethylamino)-tetracycline; 4-de(dimethylamino)-5-oxytetracycline; 4-de(dimethylamino)-7-chlortetracycline; 7-chloro-6-demethyl-4-dedimetylaminotracycline; 6-o-deoxy-5-hydroxy-4-dedimethylaminotetracycline; 6-alpha-obenzylthiomethylenetetracycline; 4-de(dimethylamino)-5-oxytetracycline; 4-de(dimethylamino)-7-chlortetracycline; 4-hydroxy-4-dedimethylaminotetracycline; 6-alpha-deoxy-5-hydroxy-4-dedimethylaminotetracycline; 4-de(dimethylamino)-tetracycline; 4-de(dimethylamino)-7-chlorotetracycline; 7-chloro-6-demethyl-4-dedimethylaminotetracyclinededimethylaminotetracycline; 6-alpha-benzyl-thiomethylenetetracycline; 11-alpha-chlortetracycline; 6-demethyl-6-deoxy-5-hydroxy-6-methylenetetracycline; 6-fluoro-demethyltetracycline; and the salts, conjugates, derivatives and combinations of these compounds.

The list of chemically modified tetracyclines is not intended to b exclusive as one skilled in the art may use minocycline as a model in preparing related analogs that would be expected to have, similar activity.

Pharmaceutical Formulations

Pharmaceutical compositions containing the form in which minocycline is to be provided are preferably administered parenterally, intraperitoneally or intramuscularly. Pharmaceutical forms suitable for injection include sterile aqueous solutions or dispersions for extemporaneous preparation of the solutions or dispersions. In all cases the form must be sterile and must be fluid to the extent that easy, syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained by the use of a coating such as lecithin, by the maintenance of the required particle size in case of adispersion and by the use of surfactants. The prevention of the action of microorganisms can be effected by various antibacterial and antifungal agents such as parabens, chlorobutanol, phenol, sorbic acid, thimerosal and the like. In many cases, isotonic agents may be included, for example, sugars or sodium chloride. Prolonged absorption of the injectable compositions can be brought about by the use in the compositions of agents delaying absorption, for example, aluminum monostearate and gelatin.

Therapeutic compositions are contemplated for use with the disclosed minocycline agents. Such compositions include comprise pharmaceutically acceptable carriers. Carrier refers to any substance suitable as a vehicle for delivering a nucleic acid molecule of the present invention. As used herein, a “carrier” refers to any substance suitable as a vehicle if delivering the minocycline of the present invention to a suitable in vivo or in vitro site. As such, carriers can act as a pharmaceutically acceptable excipient of therapeutic compositions.

Examples of carriers include, but are not limited to water, phosphate buffered saline, Ringer's solution, dextrose solution, serum containing solutions, Hank's solution, aqueous physiologically balanced solutions, oils, esters and glycols. Aqueous carriers may contain suitable auxiliary substances required to approximate the physiological conditions of the recipient, for example, by enhancing chemical stability and isotonicity.

Suitable auxiliary substances include, for example, sodium acetate, sodium chloride, sodium late, potassium chloride, calcium chloride, phosphate buffers, Tris buffers, and bicarbonate buffers. Auxiliary substances may also include preservatives, such as thimerosal, m- and o-cresol, formalin and benzyl alcohol. Preferred auxiliary substances for aerosol delivery include surfactant substances non-toxic to an animal; for example, esters or partial esters of f acids containing from about six to about twenty-two carbon atoms. Examples include; caproic; octanoic, lauric, palmitic, stearic, linoleic, linolenic, olesteric, and oleic acids. Therapeutic compositions of the present invention may be sterilized by conventional methods and/or lyophilized.

Sterile injectable solutions are prepared by incorporating the active compounds in the required amount in the appropriate solvent with various of the other ingredients enumerated above, as required, followed by filtered sterilization Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Upon formulation, solutions will be administered in a manner compatible with the dosage formulation and in such amount as is therapeutically effective. The formulations are easily administered in a variety of dosage forms preferably as injectable solutions. For the disclosed compositions, dosages in the range of 0.5 to about 25 mg/kg per day is expected to cover the range of effective dosages for minocycline, care being taken not to exceed toxic levels. In this respect, it is believed that the appropriate dose will generally be in the range of 1-10 mg/kg per day with most dosages being about 4 mg/kg/day.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of, skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In preferred embodiments, treatments employ minocycline formulations, but other closely related tetracyclines could be used; e.g., oxytetracycline, tetracycline, demeclocycline, methacycline, or doxycycline either alone, or perhaps in combination with mitocycline.

Particularly in HIV-infected and AIDS patients, there may be other medical conditions that can be treated concurrently; for example, optionally, depending on the condition of the patient, it may be desirable to include an anti-inflammatory agent in the formulation. HIV-infected patients are subject to opportunistic infections of a Wide range, including bacterial and fungal. Thus a steroidal or non-steroidal anti-inflammatory may be included in the formulation. Exemplary and well-known anti-flammatory agents include Dalfon®, Difluisal, Dolobid®, fenoprofen, Meclomen®, Prostel®, Accolate®, singulair, zyflo, advair, aerobid, azmacort, flovent, pulmicoar, qvar, intal, tilade, prednisone, prednisolone and methyl prednisolone. Over-the-counter antiflammatory agents may also be added or co-administered, including aspirin.

Antibiotics likewise may be optionally included in the formulations, including aminoglycosides, cephalosporins, clindamycin, macrolides, metronidazole, penicillins, quinolones, tetracyclines, trimethoprim, sulfamethazole, sulfonamides and vancomycin. Examples are bactrim, cipro, coxycycline erydiromycin, macrobid, and cephalexin.

Other agents may also be included in the formulations, such as reverse transcriptase inhibitors including AZT, ddI, d4T, 3TC, FTC, DAPD, 1592U89, CS92 and ddC; TAT antagonists such as Ro3-3335 and Ro24-7429; protease inhibitors such as saquinavir, ritonavir, indinavir or Viracept; agents such as acyclovir, ganciclovir or penciclovir, interferon; e.g., alpha-interferon or IL-2, immune modulation agents including bone marrow or lymphocyte transplants or other medications such as levamisol or thymosin which would increase lymphocyte numbers and/or function.

Antifungals may in certain cases be advantageous to include with the minocycline compositions. Antifungals are generally in the class of azole, polyene, allylamine or antimetabolites and include amphotericin B, nystatin, fluconazole, itraconazole, imidazole, ketoconazole, naftifine, terbinafine, 5-fluorocytosine, Griseofulvin, potassium iodide, hydroxytolbutamide, 7-hydroxy-4-trifluoromethyl coumarin, lauric acid, mephenyloin and various derivatives of these compounds.

Primate model for asymptomatic acute SIV infection. To identify events during acute and asymptomatic infection that lead to CNS disease a SIV/macaque model was developed in which the vast majority of infected animals develop CNS disease. Such a model was achieved by inoculating pigtailed macaques with a neurovirulent molecularly cloned virus, SIV/17E-Fr, and a virus swarm, SIV/DeltaB670 (Zink, et al., J. Virol., 73: 10480-10488 (1999). This model exhibits the classical stages (acute, asymptomatic and terminal) of HIV/SIV infection on an accelerated schedule, with over 90% of macaques developing inflammation in the CNS by 84 days post inoculation (p.i.). Analysis of early events in the CNS indicated that acute (10 days, p.i.) virus replication was accompanied by activation of macrophages and microglia. Virus replication and macrophage activation markers in the CNS declined in all macaques by 21 days p.i., despite high level virus replication in the peripheral blood at this time. These findings indicated the presence of mechanisms in the CNS that quell SIV replication and macrophage activation early after infection.

Levels of viral DNA in the CNS were unchanged between 10 and 21 days p.i., indicating that virus-infected cells-we're not eliminated from the brain during this transition from acute to asymptomatic infection. During late infection (56 to 84 days p.i.) increased expression of macrophage and astrocyte activation markers and increased infiltration of macrophages and cytotoxic lymphocytes accompanied resurgent virus replication in the brain and the development of encephalitis (Zink, et al., J. Virol., 73: 10480-10488 (1999). Macaques with the most severe neurological lesions exhibited the most precipitous declines in CD4+ cell counts, the highest CSF viral load and the greatest expression of viral rRNA and protein in the brain.

Identification of viral and cellular mechanisms in the CNS that control virus replication after acute infection, maintain latency dining the asymptomatic stage and mediate resurgence of virus replication and development of MV-associated encephalitis during late infection was important in discovering that minocycline could be used to prevent HIV replication in the CNS. The dynamic signaling pathways that are activated by virus replication and activated/infiltrating macrophages and lymphocytes as well as the cytokines, chemokines and neurotoxic products they elaborate, may interfere with the homeostatic signaling pathways that maintain normal quiescent brain function. It was important to understand how specific CNS cells responded to the inflammatory environment because it seemed possible that activation of specific signaling pathways in the CNS may help control local virus replication and/or mediate the development of neurological lesions. Although a stogie in vivo study reported dysregulation of protein kinases in CD4+ T-cells derived from peripheral blood during SW infection (after achieving the viral load set point), no studies had reported examination of dysregulation of intracellular signaling pathways during HIV or SIV infection of the CNS.

Using an accelerated, consistent model of HIV-associated CNS disease, activation of three different mitogen-activated protein kinases (MAPKs), ERK, JNK and p38 were quantified in the CNS during acute, asymptomatic and terminal infection. MAPK signal transduction cascades are commonly activated in response to diverse stimuli including cytokines, chemokines, cell-cell contact, matrix proteins, stress, growth factors, and viral proteins (Popik, et al. Virology, 276:1-6 (2000). Activation of ERK MAPK is typically associated with events promoting cell growth and differentiation, while activation of JNK and p3g MAPK are associated with growth arrest, apoptosis, and oncogenic transformation. Results indicated that increased activation of ERK occurs predominately in astrocytes and is present as early as 10 days p.i. Because no significant changes in JNK or p38 activation are observed at this time, the homeostatic balance between ERK and JNK/p38 activation in the CNS favors activation of ERK pathways. This response immediately precedes control of acute SIV replication and downregulation of proinflammatory responses, which occur between 10 and 21 days p.i. During terminal infection (56 to 84 days p.i.), a period characterized by resurgent virus replication and the development of neurological lesions, the balance of MAPK activation is markedly shifted in favor of JNK/p38 pathways, reflecting a failure to sustain critical signaling mechanisms in the CNS.

CNS disease is characterized by infiltration and activation of macrophages/microglia, production of proinflammatory cytokines, expression of proapoptotic and neurotoxic mediators and neuronal loss. The efficacy of memantine, CPI-1189, selegiline and nerve growth factor are being examined in clinical trials as neuroprotective agents for HIV-infected individuals (Schifitto, et al., Neurology, 57:1313-1316 (2000). These therapies were designed to inhibit the downstream effects of proinflammatory mediators or to augment neuronal function, but no single agent has emerged as the solution to both the inflammatory and neurodegenerative effects of HIV in the CNS.

Si, et al. (Society for Neuroscience, Washington, D.C., 2002) showed that minocycline inhibited HIV-1 replication in vitro in several types of cells, including macrophages, microglial cells, PBLs and astrocytes. Other studies have demonstrated minocycline's protective effects in animal models of amyotrophic lateral sclerosis, multiple sclerosis, Parkinson' disease, Huntington's disease and ischemic/traumatic brain injury (e.g., Zhu, et al., Nature, 417: 74-78 (2002)). There was, however, no evidence that minocycline would be expected to protect against HIV-induced CNS disease, nor that it would be effective in vivo.

It was reasoned that minocycline would play a dual neuroprotective role in SIV-infected macaques by inhibiting pathologic activation of p38, thus reestablishing a balance between pro- and anti-apoptotic pathways. The integrated in vivo and in vitro studies determining the efficacy of minocycline in protecting the CNS from SIV-induced damage have led to the present invention.

The SIV infection of macaques provided a basic model to investigate the cellular mechanisms that control acute virus replication in the CNS and lead to neurological disease in HIV-infected people. SIV-infected macaques develop ADDS and neuropathological changes similar to those of HIV-infected individuals, including motor and cognitive deficits as well as multifocal perivascular aggregates of brain macrophages and multinucleated giant cells, which serve as the major hosts for productive replication of virus in the CNS. However, this accepted model was modified in a manner to better determine how HIV infection in humans would lead to CNS diseases such an encephalitis.

To identify events during acute and asymptomatic infection that lead to CNS disease in a SIV/macaque, a model was needed in which the vast majority of infected animals develop CNS disease. Such a model was developed by inoculating pigtailed macaques with a neurovirulent molecularly-cloned virus, SIV/17E-Fr, and a virus swarm, SIV/DeltaB670.1 This model exhibited the classical stages (acute, asymptomatic and terminal) of HIV/SIV infection on an accelerated time schedule, with over 90% of macaques developing inflammation in the CNS by 84 days postinoculation (p.i.). Analysis of early events in the CNS indicated that acute (10 days p.i.) virus replication was accompanied by activation of macrophages and microglia. Virus replication and macrophage activation markers in the CNS declined in all macaques by 21 days p.i., despite high levee virus replication in the peripheral blood at this time. These findings indicated the presence of mechanisms in the CNS that quell SIV replication and macrophage activation early after infection.

Importantly, levels of viral DNA in the CNS were unchanged between 10 and 21 days p.i., indicating that virus-infected cells were not eliminated from the brain during this transition from acute to asymptomatic infection. During late infection (56 to 84 days p.i.) increased expression of macrophage and astrocyte activation markers and increased infiltration of macrophages and cytotoxic lymphocytes accompanied resurgent virus replication in the brain and the development of encephalitis. Macaques with the most severe neurological lesions exhibited the most precipitous declines in CD4+ cell counts, the highest CSF viral load and the greatest expression of viral RNA and protein in the brain.

Identification of viral and cellular mechanisms in the CNS that control virus replication after acute infection, maintain latency during the asymptomatic stage and mediate resurgence of virus replication and development of HIV-associated encephalitis during late infection is critical. The dynamic signaling pathways that are activated by virus replication and activated/infiltrating macrophages and lymphocytes and the cytokines, chemokines and neurotoxic products they elaborate may interfere with the homeostatic signaling pathways that maintain normal quiescent brain function. It is essential to understand how specific CNS cells respond to the inflammatory environment because activation of specific signaling pathways in the CNS may help control local virus replication and/or mediate the development of neurological lesions. To date, although one in vivo study has reported dysregulation of protein kinases in CD4+ T cells derived from peripheral blood during SIV infection (after achieving the viral load set point), no studies have examined dysregulation of intracellular signaling pathways during HIV or SIV infection of the CNS.

Using an accelerated, consistent model of HIV-associated CONS disease, activation of three different-mitogen-activated protein kinases (MAPKs), ERK, JNK and p38, were quantified in the CNS during acute, asymptomatic and terminal infection. MAPK signal transduction cascades are commonly activated in response to diverse stimuli including cytokines, chemokines, cell-cell contact, matrix proteins, stress, growth factors, and viral proteins. Activation of ERK MAPK is typically associated with events promoting cell growth and differentiation, while activation of JNK and p38 MAPK are associated with growth arrest, apoptosis, and oncogenic transformation.

The disclosed results indicate that increased activation of ERK occurs predominantly in astrocytes and is present as early as 10 days p.i. Because no significant changes in JNK or p38 activation were observed, the homeostatic balance between ERK and JNK/p38 activation in the CNS favors activation of ERK pathways. This response immediately precedes control of acute SIV replication and downregulation of proinflammatory responses, which occur between 10 and 21 days p.i. During terminal infection (56 to 84 days p.i.), a period characterized by resurgent virus replication and the development of neurological lesions, the balance of MAPK activation is markedly shifted in favor of JNK/p38 pathways, reflecting a failure to sustain critical signaling mechanisms required to maintain homeostasis in the CNS.

Materials and Methods

Animals. Twenty-eight pigtailed macaques (Macaca nemestrina) were intravenously inoculated with a virus swarm, SIV/DeltaB670, and a cloned, neurovirulent virus, SIV/17Efr. Samples of brain tissue (basal ganglia and adjacent subcortical white matter) from macaques euthanized at 10 (n=6), 21′ (n=5), 56 (n=5) and 84 (n−=12) days p.i., and from uninfected control macaques (n=3 to 8) were fixed and embedded and 5 gm sections were cut for immunohistochemical analysis of MAPKs. Of the macaques euthanized at 84 days p.i., one had no morphologic signs of encephalitis, two had mild encephalitis, five had moderate encephalitis and four had severe encephalitis based on morphologic criteria.

Quantitative immunohistochemical analysis. Monoclonal anti-active ERK antiserum (recognizes only dual phosphorylated ERK-1/2 isoforms) was obtained from Santa Cruz Biotechnology, Inc. (Santa Cruz, Calif.). Polyclonal anti-active JNK antibody (recognized only phosphorylated JNK and is reactive against the pTPpY motif present in all activated isoforms of JNK) was obtained from Promega (Madison, Wis.). Monoclonal anti-active p38 antiserum, which recognizes only dual phosphorylated p38 (all isoforms) was obtained from Sigma (St. Louis, Me.). The specificity of each antiserum for activated NAKs in pigtailed macaques was confirmed by Western blot analysis of primary macaque macrophages that had been treated with LPS for 15 min since LPS activates ERK, JNK and p38 MAPK. Brain sections from uninfected macaques and SIV-infected macaques euthanized at 10, 21, 56 and 84 days p.i. were stained immunohistochemically with each of the above antibodies, and staining was quantified in a 6 mm² section of brain from the same location in each macaque by digital image analysis.

To identify the cells that expressed activated ERK, JNK and p38, immunohistochemically stained tissues were double-labeled with KP-1 (Dako; Carpenteria, Calif.), which recognizes CD68 to identify macrophages/microglia, anti-GFAP to identify astrocytes and anti-neuron-specific enoIase (NSE; UBI, Lake Placid N.Y.) to identify neurons. Activation of MAPK in endothelium was determined morphologically. Viral antigen was identified with an anti-gp41-monoclonal antibody (kk41) obtained from the AIDS Reagent Program.

Statistical analysis. Statistical comparisons between two groups were made using the Student's two sample t-test. This test compares the means from each group while taking into consideration me respective levels of variation (i.e., the standard deviations). Small p-values (e.g., p<0.05) indicate that the data in the two samples were not likely to have been drawn from the same population; that is, the two groups are not similar. To quantity the changes in marker value, compared to the control group, percent change (% A) was calculated (e.g., (day 90−control)/control). These changes were designated positive or negative percent change for each marker whereby no change was represented by the value 1.0. The net effect of changes in all markers was then calculated as:

Net Response=−[% Δ(ERK)]−[% Δ(JNK)]+[% Δ(p38)]

representing the protective effect of either positive percent change in pERK or negative percent change for pJNK and p38. The change in net response was presented as protective being greater than zero and detrimental being less than zero.

### EXAMPLES

### Example 1

Minocycline reduces the incidence and severity of HIV encephalitis. Five of 11 SIV-infected macaques were treated with minocycline (4 mg/kg/day) beginning at 21 days post inoculation (p.i.). At this time acute virus replication, which is established by 10 days p.i., and the concurrent inflammatory responses have subsided, CNS disease has become asymptomatic, and viral RNA in brain is below the limit of detection by real-time RT-PCR.16 Oral administration of 4 mg/kg/day was selected as a therapeutic, nontoxic long-term dose of minocycline (Yen, et al., J. Dent. Res., 54:423 (1975)).

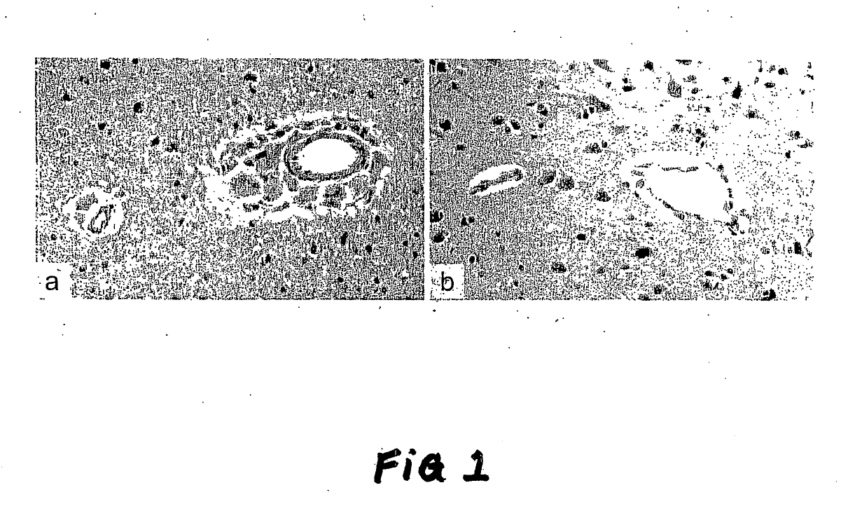

All macaques were euthanized at 84 days p.i. and their brains examined for pathological lesions typical of SIV encephalitis. In the untreated control group, only one macaque did not develop encephalitis whereas the remaining five had moderate to severe encephalitis (Table 1, FIG. 1A). In contrast, three minocycline-treated animals did not develop encephalitis, and the remaining two macaques had only mild encephalitis (Table 1, FIG. 1B). This decreased incidence of moderate and severe encephalitis in the minocycline-treated macaques was statistically significant (p=0.015). These results demonstrated the efficacy of minocycline as a neuroprotective agent in a macaque model of highly pathogenic SIV infection.

TABLE 1
Incidence and severity of encephalitis in
minocycline-treated and untreated macaques.

Severity of Encephalitis

| Treatment | None | Mild | Moderate | Severe |
|---|---|---|---|---|
| Untreated (n − 6) | 1 | 0 | 2 | 3 |
| Minocycline- | 3 | 2 | 0 | 0 |
| treated (n = 5) | | | | |

### Example 2

Minocycline suppresses CNS inflammation. The effect of minocycline on several inflammatory events characteristic of HIV/SIV encephalitis including infiltration and activation of macrophages, immunohistochemical analysis was determined on brain sections using antibodies that detect MHC Class II antigens, macrophage/microglia, cytotoxic granules found in CD8+ lymphocytes and NK cells (TIA-1) and phosphorylated (activated) p38.

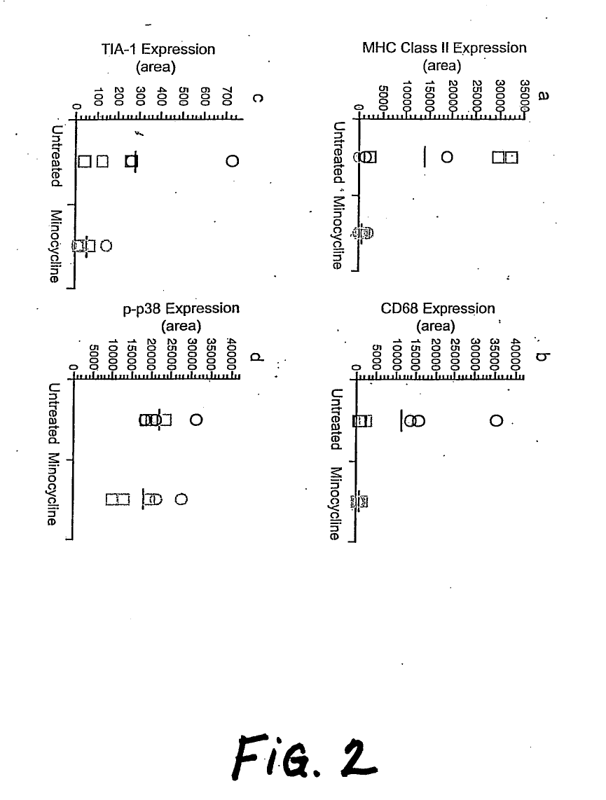

Minocycline significantly reduced expression of MHC Class II antigens (p=0.028), indicating suppressed activation of macrophages and/or endothelial cells in the brain. (FIG. 2A). Decreased infiltration and activation of macrophages in minocycline-treated animals was confirmed by significantly reduced expression of CD68 (p=0.043; FIG. 2B). Minocycline also reduced the infiltration of CD8+ and/or NK cells from the periphery into the brain as evidenced by significantly less expression of TIA-1 (p=0.034; FIG. 2C).

The effects of minocycline were not strictly limited to macrophages and lymphocytes as suggested by the trend toward reduced activation of p38 in the brain of minocycline-treated macaques (p=0.27; FIG. 2D). These data indicate collectively that minocycline exhibits both anti-inflammatory and neuroprotective properties in this SIV model of HIV CNS disease.

### Example 3

Minocycline suppresses MCP-1 expression in the CNS. To examine the mechanism(s) by which minocycline suppresses CNS inflammation, expression of the pro-inflammatory chemokine MCP-1 in CSF was sampled regularly throughout infection and in brain tissue at necropsy. MCP-1 is a pivotal chemokine mediating infiltration/activation of macrophages and to a lesser extent inflammatory T-lymphocytes into the brain; increased expression of MCP-1 has been definitively linked to pathogenesis of HIV and SIV CNS disease.

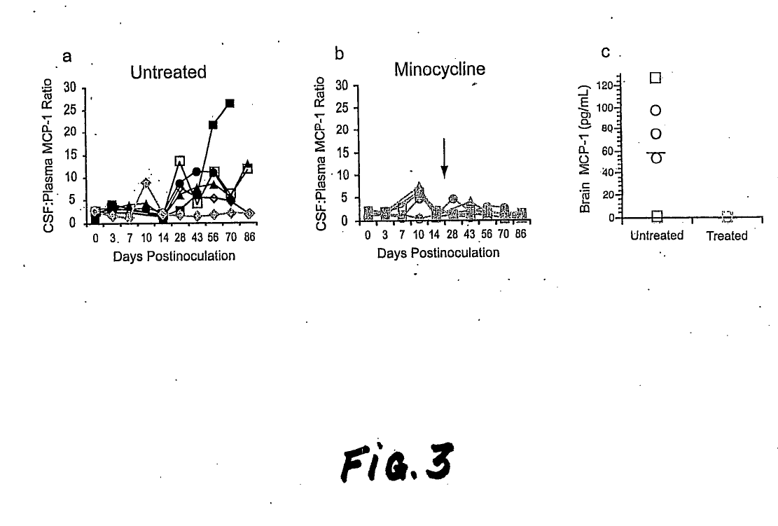

To determine whether there was a gradient of chemokine expression between the brain and periphery, which would result in an influx of macrophages to the brain, MCP-1 levels were expressed as the ratio of MCP-1 in the CSF versus that in the plasma. MCP-I ratios of many treated and untreated SIV-infected macaques increased during acute infection, peaked prior to day 14 p.i., and then declined. However, whereas the CSF:plasma MCP-1 ratios of most of the SIV-infected untreated macaques again rose after 28 days p.i., MCP-1 ratios in minocycline-treated macaques remained significantly lower (FIGS. 3A, 3B; p=0.001). At terminal infection, all minocycline-treated macaques had MCP-1 ratios that were well below 2.65, previously shown to be characteristic of animals without significant SIV encephalitis. The low terminal levels of MCP-1 in CSF were confirmed by measuring MCP-1 protein levels in brain homogenates by ELISA. Again, the minocycline-treated macaques had significantly lower levels of MCP-I protein than the SIV-infected, untreated macaques (p=0.032; FIG. 3C).

### Example 3

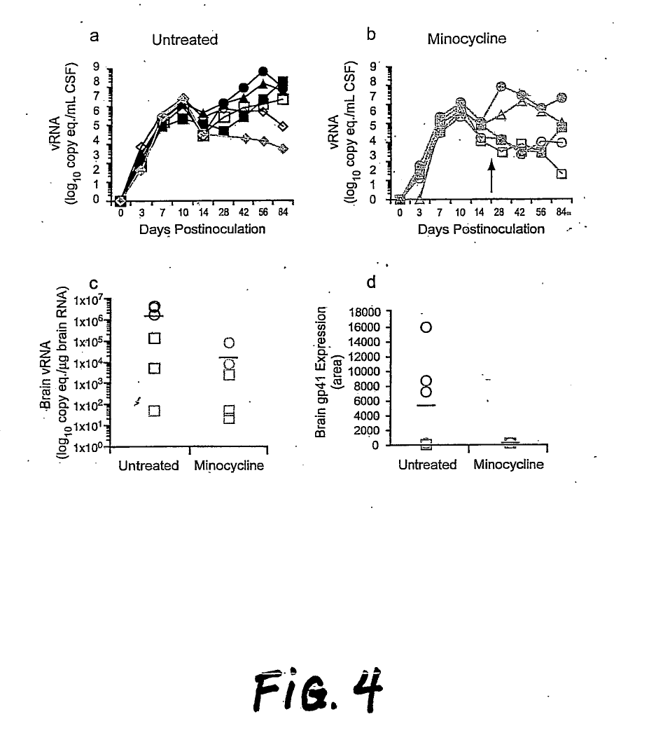

Minocycline suppresses SIV replication in the CNS. The effect of minocycline on SIV replication in the CNS of SIV-infected minocycline-treated and untreated macaques was examined by quantifying virion-associated viral RNA in the CSF and viral RNA expression in the brain by real-time RT-PCR. Minocycline significantly suppressed levels of virion RNA in the CSF and viral RNA in basal ganglia (FIGS. 4A-C; p=0.05 and 0.039, respectively). The suppressive effect of minocycline on SIV replication was further confined by quantitative image analysis on brain sections stained immunohistochemically using an antibody that recognizes SIV gp41. Again, minocycline-treated macaques had significantly lower expression of viral antigen than the SIV-infected untreated macaques (FIG. 4D; p=0.041).

### Example 4

Minocycline suppresses HIV and SIV replication in primary PBL and macrophages by p38-dependent and p38-independent mechanisms. Based on the observation that minocycline suppressed SIV replication in the CNS, macrophages and lymphocytes were examined to determine whether or not minocycline also inhibited SIV and HIV replication in these productively HIV-infected predominant target cells. In vitro doses of minocycline were chosen based on published reports demonstrating neuroprotective efficacy in rat neural cultures and based on empirical studies verifying the ability of these doses to inhibit nitric oxide-induced activation of p38 in primary macaque macrophages. The HIV strains used in these experiments were HIVBaL (for macrophage infection) and HIVIIIB (for lymphocyte infection); the SIV strains were SIV/17E-Fr and SIV/DeltaB670 (both grow well in primary macrophages and lymphocytes.).

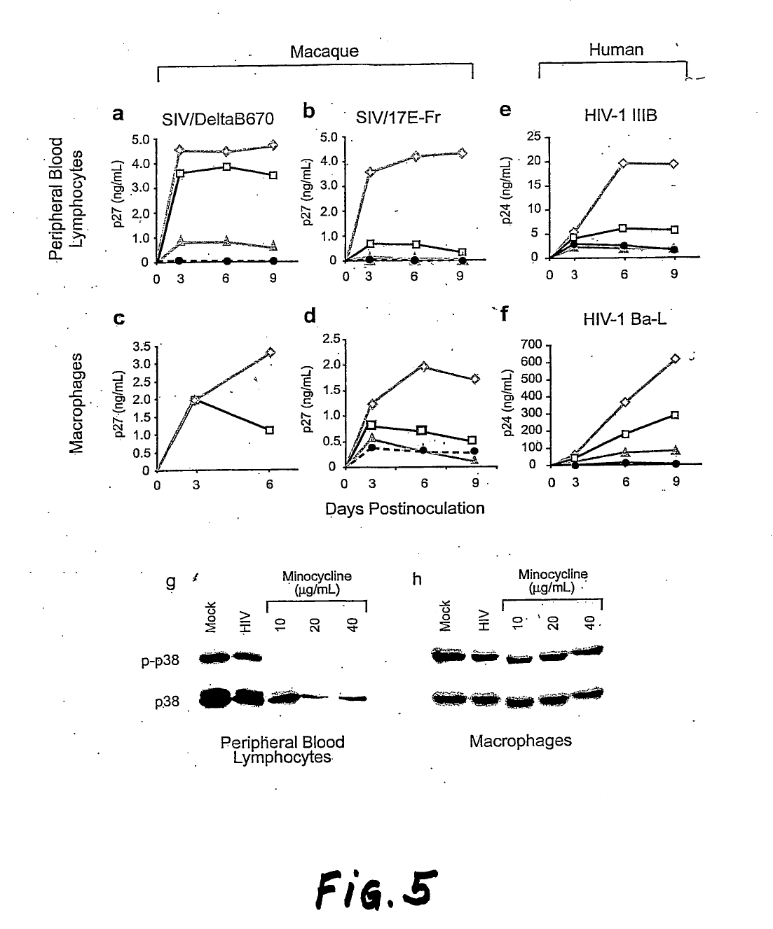

All cultures were pretreated with minocycline 24 hr prior to infection. Virus replication was quantified over time by p24 (HIV) or p27 (SIV) ELISA. Minocycline substantially inhibited HIV and SIV replication in primary lymphocytes (FIGS. 5A, 5B and 5E) and macrophages (FIGS. 5C, 5D, 5P), in a dose-dependent manner, which was evident as early as day 3 of culture in PBLs and day 6 in macrophages. Minocycline inhibited HIV replication by 92 and 99% and SIV replication by 99 and 85% in PBL and macrophages, respectively.

This was the first demonstration that the antibiotic and anti-inflammatory agent minocycline suppresses HIV and SIV replication.

### Example 5

Inhibition of p38. To determine the mechanism(s) involved in minocycline-mediated suppression of HIV and SIV replication, activation of p38 in cultures of minocycline-treated primary lymphocytes and macrophages infected with HIV was examined. p38 activation was examined because minocycline has been shown to suppress activation of p38, at least in response to proinflammatory cytokines and neurotoxic products, and because reports have suggested that activation of p38 is required for HIV replication in lymphocytes and in U1 pro-monocytic cells (Lin, et al., Neurosci. Lett. 315:61-64 (2002)).

Activation of p38 was assessed by Western blot analysis of whole cell lysates prepared at day 9 p.i. Minocycline inhibited activation of p38 (and to a lesser extent expression of p38) only in HIV-infected primary lymphocytes, but not in macrophages (FIG. 5). Similar results were obtained from SIV-infected PBL and macrophages.

It was concluded that minocycline inhibits HIV and SIV replication in primary T lymphocytes by inhibiting p38-dependent pathways and in macrophages via p38 independent mechanisms.

### Example 6

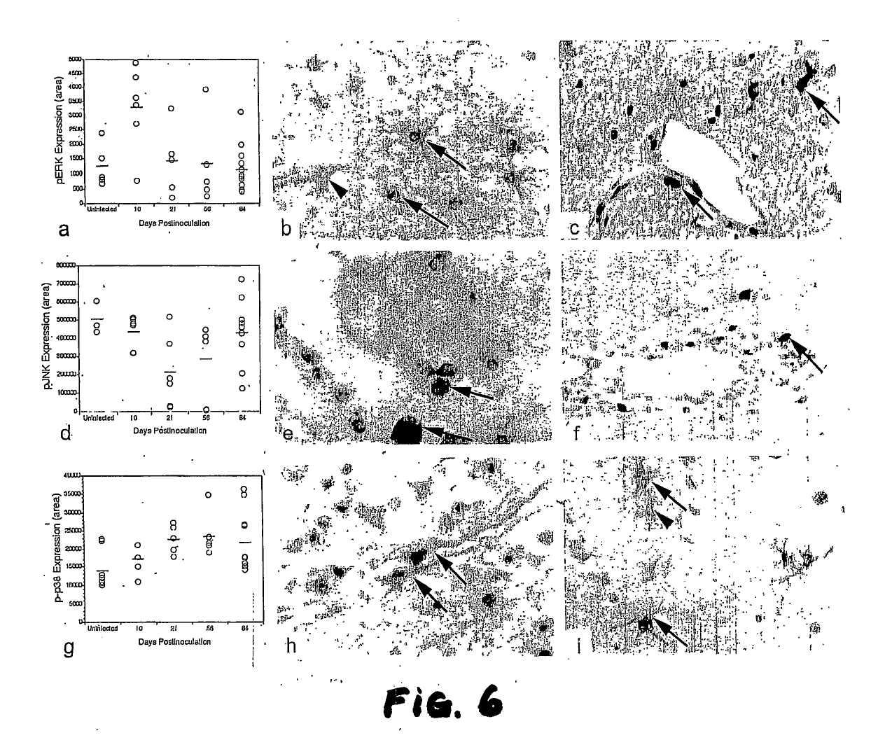

Increased activation of pERK during acute and asymptomatic infection. Expression of pERK in the brains of unidected and SIV-infected macaques euthanized at various stages of infection was quantified by digital analysis of brain sections stained immunohistochemically with antibodies specific for activated (phosphorylated) ERK (FIG. 6A). pERK expression in the brain increased significantly (p=0.019) during acute infection, peaking at 10 days p.i. Levels of pERK declined quickly from 10 to 21 days, then maintained a relatively constant level during asymptomatic infection. At terminal infection, expression of activated ERK declined further, reaching levels that were somewhat less than those in control (uninfected) macaques.

Astrocytes were the predominant cells expressing activated ERK at all time points, and astrocytes near capillaries in white matter (proximal to newly trafficking cells) were most intensely stained for pERK at 10 days p.i. (FIG. 6B). Occasional perivascalar macrophages and multinucleated giant cells also stained for pERK. There was no correlation between activation of ERK and virus infection as both infected and uninfected astrocytes and perivascular macrophages were positive for pERK (FIG. 6C), and some SrV-infected astrocytes did not stain positive for pERK.

### Example 7

Suppressed JNK activation during acute infection and rebound during terminal infection. Quantitative immunohistochemical analysis of activated (phosphorylated) JNK (pJNK) in brain sections prepared from uninfected and SIV-infected macaques indicated that JNK activation declined significantly between 10 and 21 days p.i. (p=0.04), then gradually rebounded between 21 and 84 days p.i. (p=0.048; FIG. 6). Infected macaques exhibiting moderate or severe encephalitis had significantly higher levels of JNK activation than infected macaques with no or mild encephalitis (p=0.03).

Activated JNK was expressed in neurons, macrophages and endothelium at each time point. At 56 and 84 days p.i., neuronal expression of pJNK was particularly intense. In addition, at 84 days p.i., pJNK was very prominent in cells of macrophage lineage with most perivascular macrophages expressing activated JNK (FIG. 6E). Macrophages that expressed activated JNK were not necessarily infected with SIV (FIG. 6F).

### Example 8

Increased activation of p38 after acute infection. Expression of activated (phosphorylated) p38 (p-p38) was significantly elevated at 21 p.i. (p=0.008) and remained significantly elevated throughout the remainder of the infection period, compared to uninfected controls; p<0.02; FIG. 6G). Infected macaques exhibiting moderate or severe encephalitis had significantly higher levels of p38 activation than infected macaques with no or mild encephalitis at 84 days p.i. (p=0.03).

Activated p38 was observed predominantly in neurons and astrocytes, although some staining was observed in endothelium at each time point (FIG. 6H). At 84 days p.i., the majority of neurons and astrocytes expressed activated p38, and only rare inflammatory macrophages were positive for p-p38. No correlation was observed between SIV infection of astrocytes and activation of p38.

### Example 9

Comparison between protective ARK) and degenerative (JNK and p38) MAPK activation in the CNS from acute through terminal SIV infection. Activation of ERK is typically associated with cell survival and neuroprotective events, whereas activation (above basal levels) of JNK and p38 is typically associated with apoptosis and neurodegeneration. To determine the “net” phenotype with regard to neuroprotective or neurodegenerative MAPK potential at each time p.i., levels of pERK, pJNK and p38 in SIV-infected macaques during acute (10 days p.i.), asymptomatic (21 and 56 days p.i.) and terminal (84 days. p.i.) infection were expressed as percent change from levels in uninfected macaques (n=3). The change in activation of the survival/neuroprotective MAPK, ERK was compared with the change in activation of the pro-apoptotic/neurodegenerative MAPKs JNK and p38. During acute infection (10 days p.i.), when there is active virus replication and increased expression of macrophage activation markers in the brain, protective pERK expression predominated. During the asymptomatic stage of infection (21 and 56 days p.i.), pERK still predominated although less so and the overall change in activation of protective to degenerative MAPKs more closely resembled that observed in uninfected macaques. However, at terminal infection (84 days p.i.), sustained activation of p38, rebounded activation of JNK and a marked decline in activation of ERK resulted in a net response in which the neurodegenerative MAPKs predominated. Collectively, these data showed that early in infection the balance of survival and pro-apoptotic signals in the brain favors activation of neuroprotective ERK pathways. By terminal infection there is dysregulation of these signaling molecules with a net increase in activation of the proapoptotic/neurodegenerative MAPKs, JNK and p38.

### Example 10

Inhibition of SIV Virus in vitro. Minocycline inhibits replication of two strains of SIV (SIV/17E-Fr and SIV/DeltaB670) in macrophages and microglia, as determined by p27 release. Cells were treated with minocycline either 1 hour prior to or at the same time as infection with SIV/17E-Fr or SIV/DeltaB670. Supernatants were collected at 6 and 9 days after infection and the capsid protein p27 was quantified by antigen capture.

There was a 98 percent reduction in SIV p27 levels at 10 days p.i. in SIV/DeltaB670-infected macrophages treated with 40 μg/ml of minocycline prior to infection.

There was a 95 percent reduction in SIV p27 levels at 10 days p.i. in SIV/DeltaB670-infected macrophages treated with 10 μg/ml of minocycline at the time of infection and an 85% inhibition of virus replication in SIV/17E-Fr-invected macrophages.

There was a 92 percent reduction in HIV p24 levels at 9 days p.i. in HIV-infected lymphocytes treated with 40 μg/ml of minocycline 24 hours prior to infection. There was a 99 percent reduction in HIV p24 levels at 9 days p.i. in HIV-infected macrophages treated with 40 μg/ml of minocycline 24 hours prior to infection.

Minocycline also reduced viral p27 levels in cultures of primary microglia isolated from the brains of pigtailed macaques with SIV encephalitis as compared to untreated controls. The results clearly show that minocycline suppresses replication of both SIV/17E-Fr and SIV/DeltaB670 in primary macaque macrophages and microglia.

### Example 11

Immunohistochemical analysis of brain sections derived from control and SIV-infected macaques. The immunohistochemical staining and analysis is shown in FIGS. A-I.

FIG. 6A is the quantitative immunohistochemical analysis of activated ERK (pERK); horizontal bars represent the means of each group, pERK expression was statistically different between control and 10 day groups (p=0.019), 10 day and 21 day groups (p=0.05) and control and 84 day groups (p=0.01)

FIG. 6B shows co-localization of pERK-(purple) and GFAP (brown) in astrocytes (arrows) next to a capillary (arrowhead) in brain from a representative SIV-infected macaque at 10 days p.i.

FIG. 6C shows co-localization of pERK (brown) and SIV gp41 (red) in macrophages (arrow's) in the brain of a representative SIV-infected macaque at 10 days p.i.

FIG. 6D is a quantitative immunohistochemical analysis of activated JNK (pJNK); horizontal bars represent the means of each group, pJNK expression was statistically different between 10 day and 21 day groups (p=0.04) and 21 day and 84 day groups (p=0.048).

FIG. 6E shows co-localization of pJNK (blue) and CD68 (red) in macrophages (arrows) in brain from a representative SIV-infected macaque at 84 days p.i

FIG. 6F shows co-localization of pJNK (red) and SIV gp41(blue) in a perivascular macrophage (arrow) in brain from a representative SIV-infected macaque at 84 days p.i.

FIG. 6G is a quantitative immunohistochemical analysis of activated p38 (p=p38); horizontal bars represent the means of each group, p38 expression was statistically different between control and 21 day groups (p=0.008) and control and 84 day groups (p=0.022).

FIG. 6H shows co-localization of p-p38 (red) and NSE (brown) in neurons (arrows) in brain from a representative SIV-infected macaque at 84 days.

FIG. 5I shows co-localization of p-p38 (red) and GFAP (brown) in astrocytes (arrows) in brain from a representative SIV-infected macaque at 84 days p.i. Arrowhead indicates an astrocyte that is not expressing p-p38.

### Example 12

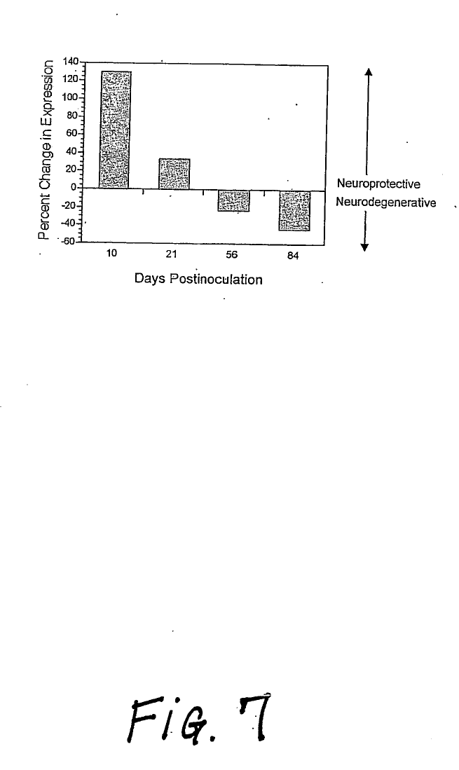

Comparative expression of pERK. FIG. 7 shows a comparative expression of pERK (neuroprotective) vs. pJNK and p-p38(neurodegenerative) in brains of macaques during acute (10 days p.i.), asymptomatic (21 and 56 days p.i.) and terminal (84 days p.i.) infection. Bars represent the net effect of neuroprotective and neurodegenerative influences based on percent change in expression of these signaling molecules, assuming that pERK, pJNK and p-p38 have equal and independent influences. During acute infection, there was a net increase in expression of pERK, whereas during terminal infection neurodegenerative pathways, particularly p-p38, predominated.

Discussion

The antibiotic minocycline not only inhibits HIV and SIV replication in vitro, but also significantly reduces the incidence and severity of encephalitis in a rigorous SIV/macaque model of HAD in which greater than 90% of infected macaques develop CNS lesions. The latter observation is particularly impressive, given the rapidity and severity of SIV encephalitis in the model and the ability of minocycline to intervene effectively during asymptomatic infection.

This is believed to be the first report demonstrating anti-inflammatory and neuroprotective activity of an antibiotic against virus infection in a model for human infection. Given that the prevalence of HAD has increased in the era of HAART, this discovery is believed to have profound implications for prevention/treatment of HAD. The ability of minocycline to prevent, increased expression of MCP-1 in the brain is unquestionably an important if not a critical mechanism mediating the neuroprotective effect in the SIV model of HIV CNS disease.

Macrophages provide a primary mode of transport for HIV/SIV into the brain, are the major sources for HIV/SIV replication in the CNS and produce toxic mediators during HIV CNS disease. The minocycline decreased activation of macrophages/microglia and influx of cytotoxic lymphocytes in SIV-infected macaques is similar to findings in rodent models of neurodegeneration; however, the present study is the first to link MCP-1 to the mechanisms mediating the neuroprotective effects of minocycline. This novel finding suggests that minocycline has broader clinical applicability to neurodegenerative disorders in which MCP-1-dependent infiltration and activation of macrophages is an important determinant of neuropathology. It is believed that minocycline treatment will prove beneficial to HIV-infected individuals who are at higher risk for development of HAD by virtue of a genetic polymorphism in the MCP-1 promoter region that increases MCP-1 levels.

An unexpected result of these studies was the ability of minocycline to substantially inhibit SIV and HIV replication in vitro. It seems unlikely that minocycline possesses classical antiviral activity as do reverse transcriptase and protease inhibitors since this antibiotic was not engineered to target a specific viral protein. Rather than exerting direct antiviral activity, minocycline may modify the intracellular environment such that it becomes non-permissive for HIV/SIV replication. Minocycline treatment initiated at 21 days p.i. significantly suppressed virus replication in CSF and brain and to a less marked extent in plasma, likely because of the high plasma vital loads (˜10⁸ copy eq./mL plasma by day 10 p.i.) in this accelerated SIV/macaque model. Even so, plasma viral RNA levels were significantly lower in the three minocycline-treated macaques that did not develop encephalitis as compared to the two treated macaques that developed mild encephalitis 0.05).

One clear advantage of the ability of minocycline to modulate cellular pathways that inhibit SIV/HIV replication is that this mechanism is expected to be comparatively resistant to evolution of virus mutations, which plague traditional antiviral therapies. Moreover, the finding that minocycline modifies intracellular environments by separate mechanisms in PBL and macrophages during suppression of HIV and SIV replication suggests that the potential development of resistance mutations to minocycline in macrophages are not expected to confer minocycline resistance in T-lymphocytes.

Minocycline is a semi synthetic second-generation tetracycline that readily crosses the blood-brain barrier. It is an inexpensive, safe and effective antibiotic that has been prescribed for years and is available in generic form. The novel findings that minocycline inhibits SIV and HIV replication in primary macrophages and lymphocytes in vitro and suppresses SIV replication in the brain and associated neuropathology in vivo, strongly support the conclusion that minocycline will provide an excellent supplement to HAART in the treatment of HAD and will be effective in maintaining low viral loads in individuals in which HAART therapy must be discontinued. An additional benefit is the antimicrobial activity of minocycline, particularly toxoplasmosis, malaria and several sexually transmitted diseases suggesting its utility for long-term use in global areas in which individuals frequently harbor multiple infections in addition to HIV.

The entire contents of all patents, published patent applications and other references cited herein are hereby expressly incorporated in their entireties by reference as if set forth in full for all that is disclosed therein.

While the various embodiments of the present invention have been described in detail, it is apparent that modifications and adaptations of those embodiments will occur to those of skill in the art. It is to be understood that such modifications and modifications are within the scope of the invention, as set forth in the following claims.

1. A pharmaceutical composition comprising a tetracycline analog or tetracycline modified compound or a physiologically acceptable salt thereof in an amount effective to suppress HIV replication in HIV/SIV-infected mammalian plasma and central nervous system (CNS).

2. The composition of claim 1 wherein the tetracycline analog is selected from the group consisting of minocycline, oxytetracycline, tetracycline, demeclocycline, methacycline, and doxycycline.

3. The composition of claim 1 wherein the HIV/SIV-infected mammal is a human exhibiting AIDS.

4. A pharmaceutical composition comprising minocycline or a physiologically acceptable salt thereof in an amount effective to suppress HIV replication in brain and peripheral locations in an HIV-infected human.

5-7. (canceled)

8. The composition of claim 1 wherein the tetracycline modified analog is a chemically modified tetracycline selected from the group consisting of: 4-dedimethylaminotetracycline, 4-dedimethylamino-oxytetracycline, 4-dedimethylamino-7-chlortetracycline, 4-hydroxy-4-dedimethylaminotetracycline, 5a,6-anhydro-4-hydroxy-4-dedimethylaminotetracycline, 6α-deoxy-5 hydroxy-4-dedimethylaminotetracycline, 6-dimethyl-6-deoxy-4-dedimethylaminotetracycline, 4-dedimethylamino-11-hydroxy-12a-deoxytetracyclins, 12a-deoxy-4-deoxy-4-dedimethylaminotetracycline, 6-alpha-deoxy-5-hydroxy-4-dedimethylaminodoxycycline, 12a,4a-anhydro-4-dedimethylaminotetracycline, minocycline-CMT, 7-dimethylamino-6-demethyl-6-deoxy-4-dedimethylaminotetracycline, 6a-benzylthiomethylenetetracycline, 2-nitrilo analogs of tetracycline (tetracyclinonitrile), mono-N-alkylated tetracycline amide, 6-fluoro-6-demethyltetracycline, 11a-chlortetracycline, tetracycline pyrazole, 12a-deoxytetracycline, 4-dedimethylamino-5-oxytetracycline, 5a,6-anhydro-4-hydroxy-4-dedimethylminotetracycline, 12a,4a-anhydro-4-dedimethylaminotetracycline, tetracyclonitrile, 7-chloro-4-dedimethylaminotetracycline, 12a-deoxy-4-deoxy-4-dedimethylaminotetracycline, 4-dedimethylamino-7-chlorotetracycline, 4-dedimethylamino-7-dedimethylaminotetracycline, the 2-nitrilo analogs of tetracycline, 4-dedimethylamino 12a-deoyotetracycline, 4-dedimethylamino-7-chlorotetracycline, 6-alpha-deoxy-hydroxy-4-dedimethylaminotetracycline, tetracyclonitrile, 6-alpha-benzylthiomethyltetracycline, 11-alpha-chlorotetracycline, 5-hydroxytetracycline, 6-demethyl-7-chlortetracycline, 6-demethyl-6-deoxy-5-hydroxy-6-methylenetetracycline, 6-alpha-benzylthiomethylenetetracycline, mono-N-alkylated tetracycline amide, 2-acetyl-8-hydroxy-1-tetracycline, 6-demethyl-6-deoxytetracycline, 6-demethyl-6-deoxy-5-hydroxy-6-methylenetetracycline, 2-acetyl-8-hydroxyl-1-tetracycline, 4-hydroxy-4-dedimethylaminotetracycline, 5a,6-anhydro-4-hydroxy-4-dedimethylaminotetracycline, 6-demethyl-6-deoxy-4-dedimethylaminotetracycline, 6-deoxy-8-demethyl-4-dedimethylaminotetracycline, 6a-deoxy-5-hydroxy-4-dedimethylaminotetracycline, pyrazole derivative of tetracycline, 7-chloro-6-demethyl-4-dedimethylaminotetracycline, 11-alpha-chlortetracycline, 4-dedimethylamino-7-chlortetracycline, 4-de(dimethylamino)-tetracycline, 4-de(dimethylamino)-5-oxytetracycline, 4-de(dimethylamino)-7-chlortetracycline, 7-chloro-6-demethyl-4-dedimethylaminotetracycline, 6-o-deoxy-5-hydroxy-4-dedimethylaminotetracycline, 6-alpha-obenzylthiomethylenetetracycline, 4-de(dimethylamino)-5-oxytetracycline, 4-de(dimethylamino)-7-chlortetracycline, 4-hydroxy-4-dedimethylaminotetracycline, 6-alpha-deoxy-5-hydroxy-4-dedimethylaminotetracycline, 4-de(dimethylamino)-tetracycline, 4-de(dimethylamino)-7-chlortetracycline, 7-chloro-6-demethyl-4-dedimethylaminotetracycline, dedimethylaminotetracycline, 6-alpha-benzyl-thiomethylenetetracycline, 11-alpha-chlorotetracycline, 6-demethyl-6-deoxy-5-hydroxy-6-methylenetetracycline, 6-fluoro-demethyltetracycline, and the salts and combinations thereof.

9. The composition of claim 1 or 4 further comprising an antiflammatory agent.

10. The composition of claim 9 wherein the anti-inflammatory agent is selected from the group consisting of Dalfon®, Difluisal, Dolobid®, fenoprofen, Meclomen®, Prostel®, Accolate®, singulair, zyflo, advair, aerobid, azmacort, flovent, pulmicoar, qvar, intal, tilade, prednisone, prednisolone and methyl prednisolone.

11-16. (canceled)

17. A method of suppressing HIV replication in a mammal, comprising administering to said mammal, an amount of minocycline or a derivative thereof effective to suppress HIV replication.

18-50. (canceled)
